(19) United States
(12) Patent Application Publication
(10) Pub. No.: US 2007/0093848 A1
(43) Pub. Date: Apr. 26, 2007

(54) Cervical drill guide apparatus

(76) Inventors: Peter Harris, Leesburg, VA (US); Larry McClintock, Gore, VA (US); Todd Wallenstein, Ashburn, VA (US)

Correspondence Address: CARTER, DELUCA, FARRELL & SCHMIDT, LLP, 445 BROAD HOLLOW ROAD, SUITE 225, MELVILLE, NY, 11747, US

(21) Appl. No.: 11/541,357

(22) Filed: Sep. 29, 2006

Related U.S. Application Data
(60) Provisional application No. 60/721,484, filed on Sep. 29, 2005.

(51) Int. Cl. A61B 17/60

(52) U.S. Cl. 606/96

(57) ABSTRACT

A cervical drill guide apparatus includes a handle assembly having a housing and an extension member attached to one end of the housing. The handle assembly includes a fixed handle and a movable handle. A distal end of the extension member includes a plurality of fingers that are radially expandable or contractible in response to relative movement between an actuation shaft and the extension tube. The fingers are adapted for releasably engaging an opening in a plate. Additionally, a guide member is releasably attached to the distal end portion of the cervical drill guide apparatus. The guide member includes at least one opening therethrough for receiving a drill or other surgical instrumentation. The extension member may be rotatable in relation to the housing.

### CROSS-REFERENCE TO RELATED APPLICATIONS

The present disclosure claims the benefit of and priority to Provisional Patent Application No. 60/721,484, filed on Sep. 29, 2005, the entire contents of which are hereby incorporated by reference.

### BACKGROUND

1. Technical Field

The present disclosure relates generally to orthopedic spinal surgery and in particular to a cervical drill guide apparatus and methods for guiding a cervical drill, bone screw, or other instrumentation during spinal surgery.

2. Background of Related Art

The spinal column is a complex system of bones and connective tissues that provide support for the human body and protection for the spinal cord and nerves. The adult spine is comprised of twenty-four vertebral bodies, which are subdivided into three areas, including seven cervical vertebrae, twelve thoracic vertebrae and five lumbar vertebrae. Between each vertebral body is an intervertebral disc that cushions and dampens the various translational and rotational forces exerted upon the spinal column.

There are various disorders, diseases, and types of injury which the spinal column may experience in a lifetime. These problems may include, but are not limited to, scoliosis, kyphosis, excessive lordosis, spondylolisthesis, slipped or ruptured discs, degenerative disc disease, vertebral body fracture, and tumors. Persons suffering from any of the above conditions typically experience extreme or debilitating pain and often times diminished nerve function.

One of the more common solutions to any of the above-mentioned conditions involves a surgical procedure known as a spinal fusion. Spinal fusion involves fusing two or more vertebral bodies together to eliminate motion at the intervertebral disc or joint. To achieve spinal fusion, natural or artificial bone, along with a spacing device, replace part or all of the intervertebral disc to form a rigid column of bone. Mechanical hardware is connected to the adjacent vertebrae to stabilize the spine in that area while the bone grows and the fusion occurs.

The mechanical hardware used to immobilize the spinal column typically involves a series of bone screws and metal rods or plates. When the spine surgery is performed posteriorly, it is common practice to place pedicle bone screws into the vertebral bodies and then connect a metal rod between the screws, thus creating a rigid structure between adjacent vertebral bodies. When the spinal surgery is performed anteriorly, it is common practice to attach a thin metal plate directly to the vertebral bodies and secure it to each vertebral level using one or more bone screws. For the remainder of this disclosure, references to spinal surgery will be referring to the anteriorly performed surgery in which a metal plate is secured directly to the vertebrae using bone screws.

Because the spine is routinely subject to mechanical loads which cycle during movement, a primary concern of physicians performing metal plate implantation surgeries, as well as of the patients in whom the implants are placed, is the risk of screw pullout. This is of particular concern in the cervical region because of the critical vessels that abut the anterior surfaces of the cervical spine. Screw pullout occurs when the cylindrical portion of the bone that surrounds the inserted screw fails. A bone screw that is implanted into the vertebrae perpendicular to the plate is particularly weak because the region of the bone that must fail for pullout to occur is only as large as the outer diameter of the screw threads. Screws which are angled inward towards one another, also referred to as “toe-nailed”, or ones which diverge within the bone have been found to greatly reduce the likelihood of screw pull out because the region of bone that must fail is increased as compared to that of screws implanted perpendicular to the plate.

The metal plates used to connect the vertebrae in spinal surgery are well known in the art. These plates may define any number of openings configured for receiving bone screws. The openings for receiving the screws may include a beveled or angled edge for more securely receiving the angled screws. The metal plates may also include openings or grooves for releasably receiving an elongated handle member for maintaining the metal plate during implantation. Because the metal plates used in spinal fusion are relatively small and awkward to handle, elongated handle members have been developed for releasably engaging the metal plates such that they may be held in position while the bone screws are being applied. The elongated handle members generally include a handle assembly for grasping the handle member and an extension member connected thereto. The distal end of the extension member may include any number of clips, protrusions, tabs or the like for releasably engaging the metal plate.

As discussed above, positioning of the bone screws used to secure the metal plate to the vertebrae is important to preventing screw pullout, and thus a successful spinal fusion. The elongated handle members may further be configured to include a guide member for guiding the drill, screws, or other instrument for assisting a surgeon in positioning the bone screws during implantation of the metal plate. Commonly owned U.S. Pat. No. 7,094,242 to Ralph et al., discloses such a device, and is incorporated herein by reference in its entirety.

Conventional drill guides generally include a handle assembly fixedly attached to the proximal end of an extension member. The distal end of the extension member generally includes an assembly configured for releasable engagement with a metal plate. The extension member may be configured to include one or more guide members. Alternative drill guides include a guide member that is independently attached to the mounting assembly. During procedures involving surgeons with different preferences, more than one surgeon or the use of multiple instruments within the surgical field, the handle assembly of the drill guide often is oriented in a less than convenient position. Because the handle assembly is fixedly attached to the extension member, the orientation of the handle assembly cannot be adjusted. Furthermore, because the guide members are affixed to a mounting plate or incorporated in the extension member, the guide members cannot be removed or replaced. In the event that the guide member malfunctions, or the procedure calls for an alternate guide member configuration, the entire drill guide must be replaced.

Therefore, it would be beneficial to have a drill guide apparatus including a handle assembly that can be selectively positioned about an extension member prior to or during implantation of a metal plate. It would further be beneficial to have a guide member that can be removably affixed to the drill guide apparatus.

### SUMMARY

A cervical drill guide apparatus according to one embodiment of the present disclosure includes a handle assembly having an extension tube extend distally therefrom. The handle assembly includes a housing with a fixed handle and a movable handle. The movable handle is pivotable with respect to the fixed handle. A biasing mechanism is located between the fixed handle and the movable handle for biasing the movable handle away from the fixed handle, thereby defining a first position. A latch is disposed on a proximal portion of the handle assembly and permits locking the fixed handle and the movable handle in a second position after a practitioner has pivoted the movable handle towards the fixed handle.

Pivotable movement of the movable handle towards the fixed handle causes an actuation shaft to move distally through the extension member. At the distal end of the extension member, a plurality of slits defines a plurality of fingers that are radially expandable in relation to the longitudinal axis of the extension member. The fingers are adapted and configured for releasably engaging an opening in a bone plate. In addition, a mount assembly is located at the distal end of the extension member. The mount assembly releasably retains a guide member. The guide member includes at least one opening therethrough for receiving a drill bit or other surgical instrument.

### BRIEF DESCRIPTION OF THE DRAWINGS

Embodiments of the presently disclosed cervical drill guide apparatus are described herein with reference to the accompanying drawings, wherein:

FIG. 1 is an isometric view of an embodiment of the drill guide apparatus of the present disclosure including a handle assembly, extension member, mounting assembly and guide member;

FIG. 2 is a cross-sectional side view of the drill guide apparatus of FIG. 1;

FIG. 3 is an exploded side view of the drill guide apparatus of FIGS. 1 and 2 including a metal plate;

FIG. 4A is a front elevational view of the drill guide apparatus of FIGS. 1-3 including a metal plate attached thereto;

FIG. 4B is an enlarged view of portion 4B of the drill guide apparatus of FIG. 4A;

FIG. 5A is a side view of the drill guide apparatus of FIGS. 1-3 in a first or open position;

FIG. 5B is a cross-sectional view of the distal end of the drill guide apparatus of FIG. 5A taken along section line 5B-5B;

FIG. 6A is a side view of the drill guide apparatus of FIGS. 1-3 in a second or closed position;

FIG. 6B is a cross-sectional view of the distal end of the drill guide apparatus of FIG. 6A taken along section line 6B-6B;

FIG. 7 is an enlarged side view of the distal end of the drill guide apparatus of FIGS. 1-3;

FIG. 8A is a side view of the guide member of FIGS. 1-4B;

FIG. 8B is an isometric view of the guide member of FIG. 8A;

FIG. 9A is a side view of an alternate embodiment of a guide member of the present disclosure;

FIG. 9B is an isometric view of the guide member of FIG. 9A;

FIG. 10A is a side view of another alternate embodiment of a guide member of the present disclosure;

FIG. 10B is an isometric view of the guide member of FIG. 10A;

FIG. 11A is a side view of yet another alternate embodiment of a guide member of the present disclosure; and

FIG. 11B is an isometric view of the guide member of FIG. 11A.

### DETAILED DESCRIPTION OF THE EMBODIMENTS

While the presently disclosed cervical drill guide apparatus will be described more fully hereinafter with reference to the accompanying drawings, in which particular embodiments are shown, it is to be understood at the outset that persons skilled in the art may modify the apparatus herein described while achieving the functions and results of this apparatus. Accordingly, the descriptions that follow are to be understood as illustrative and exemplary of specific structures, aspects, and features within the broad scope of the present disclosure and not as limiting of such broad scope. Like numbers refer to similar features of like elements throughout.

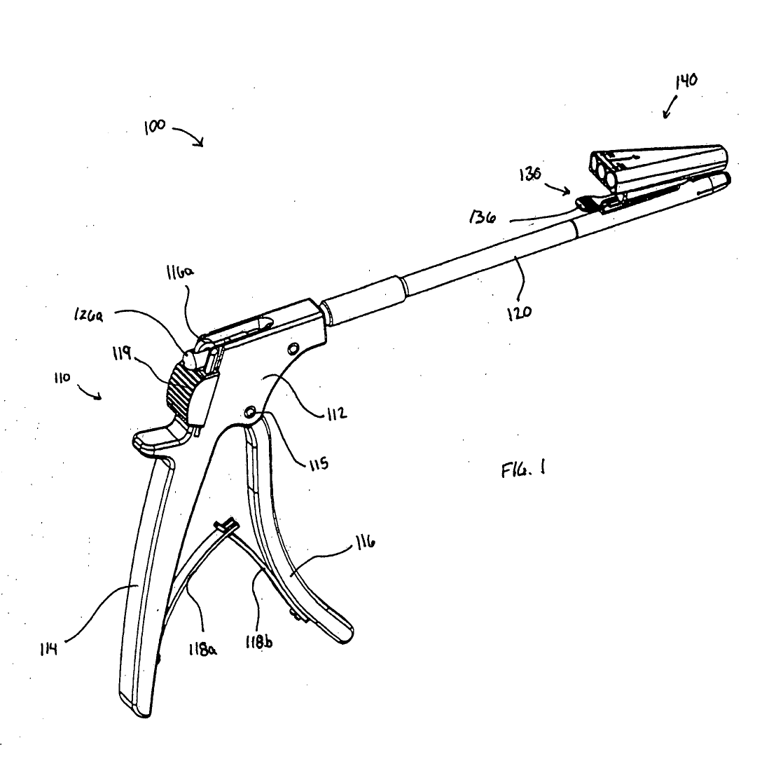

Referring to FIG. 1-3, an illustrative embodiment of the presently disclosed drill guide apparatus is illustrated therein and generally designated as drill guide 100. Drill guide 100 includes a handle assembly 110, an extension member 120, a mount assembly 130, and a removably attachable guide member 140. In this disclosure, as is traditional, the term “proximal” will refer to the end of the drill guide 100 (or other element) which is closer to the user, while the term “distal” will refer to the end which is further from the user.

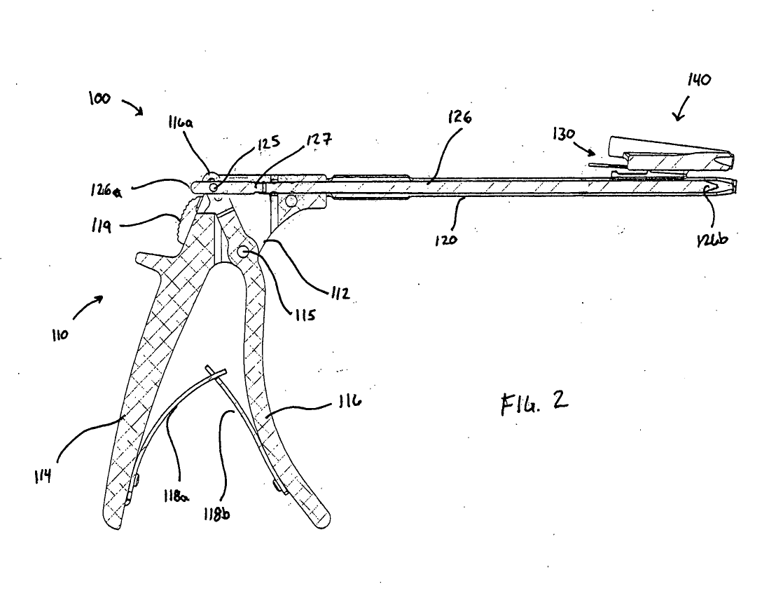

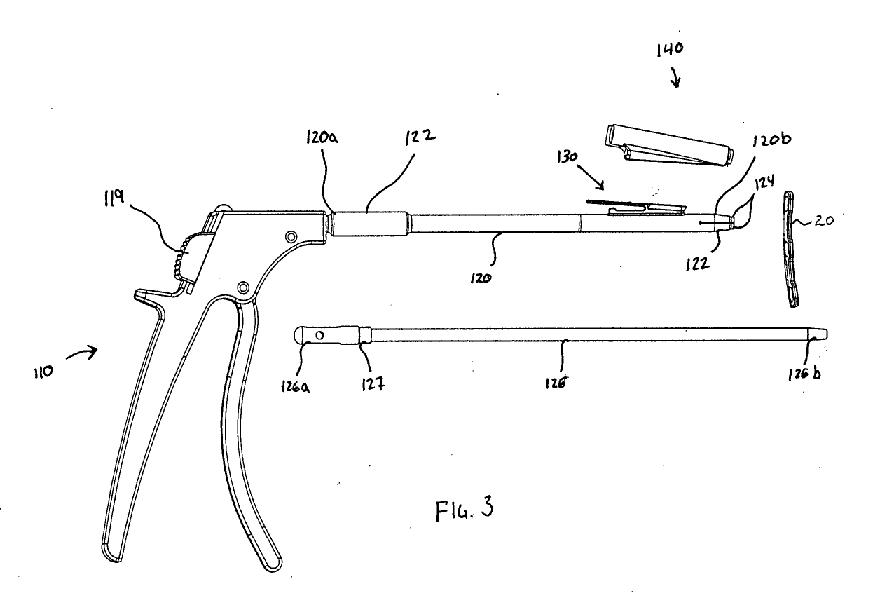

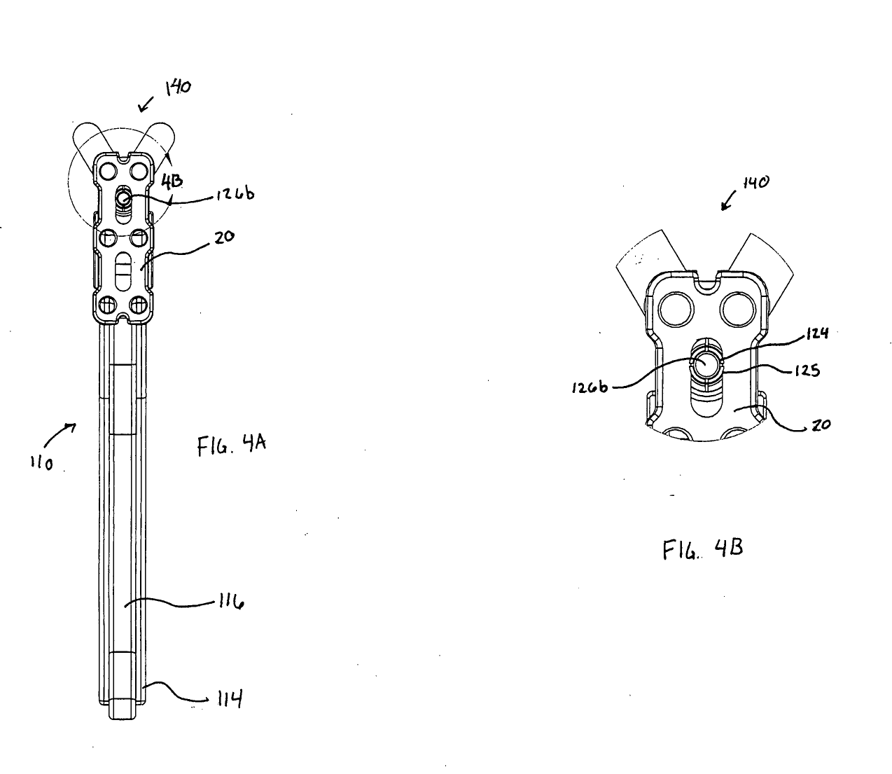

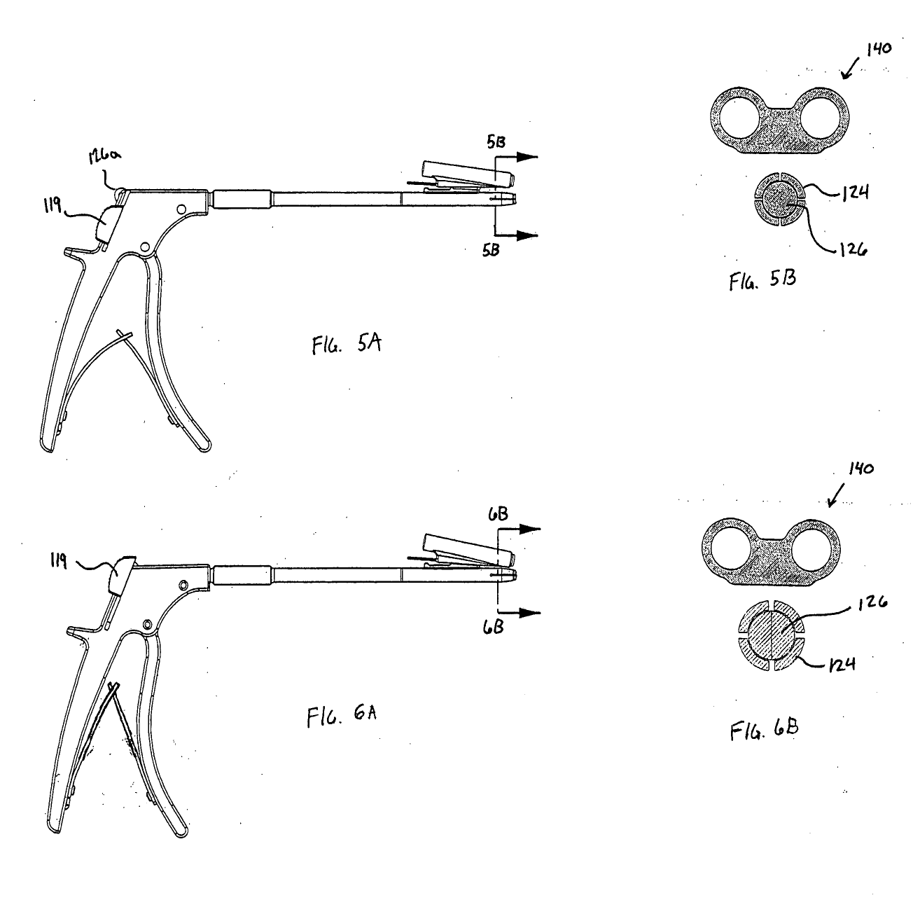

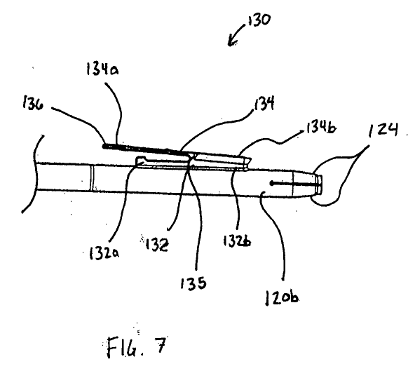

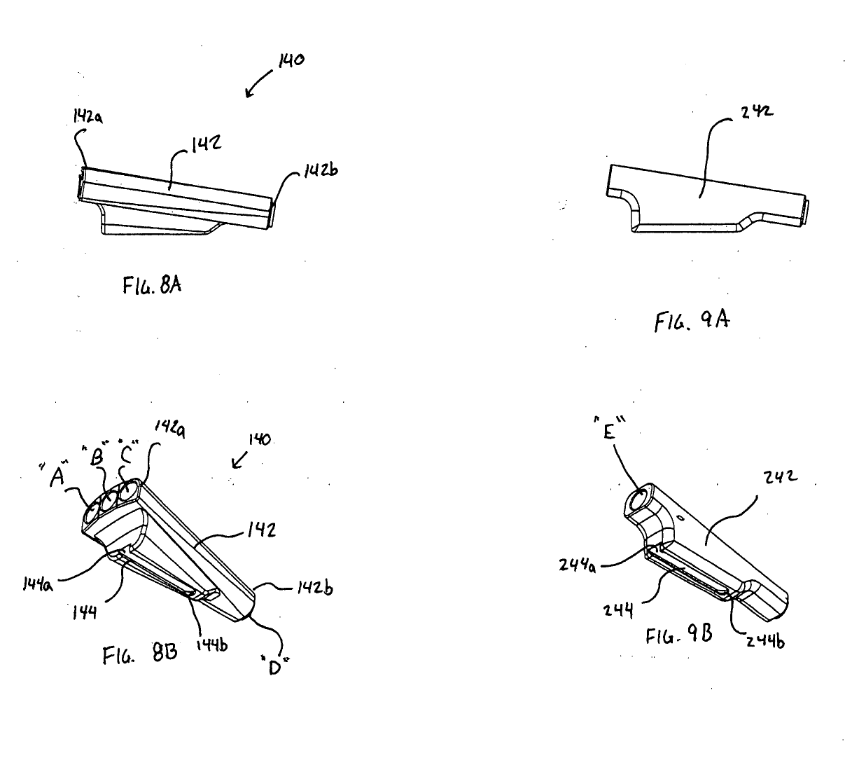

With reference to FIGS. 1 and 2, handle assembly 110 forms a pistol-like grip configured such that a surgeon may operate drill guide 100 with a one hand. Handle assembly 110 includes a housing 112, a fixed handle 114, and a movable handle 116. Movable handle 116 is pivotably connected to housing 112 by handle pin 115. Movable handle 116 includes a tip 116a located at a proximal end thereof that extends through housing 112. Tip 116a of movable handle 116 operably engages actuation shaft 126. Handle assembly 110 further includes a latch 119 slidably mounted to housing 112. Leaf springs 118a, 118b are fastened to fixed handle 114 and movable handle 116 to bias handle assembly 110 towards a first or open position, wherein movable handle 116 is spaced apart from fixed handle 114. In alternate embodiments, movable handle 116 may be biased using any conventional biasing mechanism including, but not limited to, springs, hydraulics, and pneumatics.

Referring now to FIGS. 2 and 3, actuation shaft 126 extends substantially the length of drill guide 100. Actuation shaft 126 includes a proximal end 126a and a distal end 126b. Proximal end 126a is configured to be operably received within housing 112. Proximal end 126a is further configured to engage tip 116a. Proximal end 126a engages tip 116a such that articulation of movable handle 116 about handle pin 115 towards fixed handle 114 (i.e. pivotable movement of handle 116) causes longitudinal movement of actuation shaft 126 in the distal direction through extension member 120. In the present embodiment, proximal end 126a is configured to receive tip 116a therethrough (FIG. 1). A locking pin 125 may further be used to secure proximal end 126a to tip 116a of movable handle 116. Proximal end 126a may also be configured to connect to tip 116a using mechanical fasteners and/or geared surfaces. Actuation shaft 126 extends from within housing 112 and extends to a distal end 126b located within extension member 120. Proximal end 126a further includes a collar 127 for preventing actuation shaft 126 for from over-extending distally through housing 112.

Still referring to FIGS. 2 and 3, extension member 120 is an elongated tubular member having a proximal end 120a and a distal end 120b. Proximal end 120a is rotatably mounted to housing 112. Extension member 120 includes a collar 122 for selectively rotating extension member 120 relative to handle assembly 110. Extension member 120 may be configured to rotate 360° relative to handle assembly 110. Extension member 120 may be configured with predetermined stops or positions for selectively positioning extension member 120 relative to handle assembly 110. A metal plate 20 may be releasably engaged to distal end 120b such that rotation of extension member 120 also rotates plate 20. Collar 127 may include graduations or markings for selectively aligning or positioning handle assembly 110 and extension member 120.

Extension member 120 is configured to permit the longitudinal movement of actuation shaft 126 therein. Distal end 120b is configured for releasable attachment to metal plate 20. Distal end 120b of extension member 120 includes a tapered end portion 122 defining a plurality of fingers 124 (FIG. 7) that are radially expandable in relation to extension member 120. Fingers 124 are sized to be received within one or more openings formed on metal plate 20 when fingers 124 are in a first or relaxed position. Fingers 124 include a ridge or lip 125 (FIG. 4B) for releasably engaging an opening formed in metal plate 20 when fingers 124 are in a second or expanded position (FIGS. 4A and 6B). Fingers 124 of expansion member 120 and distal end 126b are configured such that longitudinal advancement of actuation shaft 126 through expansion member 120 causes radial expansion of fingers 124 (FIG. 6B). Expansion member 120 and actuation shaft 126 are further configured such that retraction of actuation shaft 126 within expansion member 120 causes fingers 124 to return to a first or relaxed position (FIG. 5B). Thus, the disengaging drill guide 100 from metal plate 20.

Referring now to FIG. 7, distal end 120b further includes a mount assembly 130 for removably attaching guiding member 140 to drill guide 100 (FIG. 3). Mount assembly 130 includes a first member 132 and a second member 134. First and second members 132, 134 each include a proximal end 132a, 134a and a distal end 132b, 134b, respectively. First member 132 is fixedly attached to distal end 120b using any conventional means, including but not limited to adhesives, welding and fasteners. Second member 134 is pivotably mounted to first member 132. A spring mechanism 135 biases distal end 134b of second member 134 against distal end 132b of first member 132. Proximal end 134a of second member 134 may define a lever or tab 136 (FIG. 1) for effecting the separation of distal ends 132b, 134b of first and second members 132, 134, respectively, when depressed.

Second member 134 of mount assembly 130 is configured for slidably receiving guide member 140. Second member 134 may further be configured to engage first member 132 such that once guiding member 140 has been received on second member 134 guide member 140 cannot be removed without depressing lever 136. First and/or second members 132, 134 may include a coating, for example rubber, and/or be configured with ridges, grooves, or knurls that more securely retains guide member 140.

With reference now to FIGS. 3, 8A, and 8B, guide member 140 includes a base 142 having a proximal end 142a and a distal end 142b. Base 142 defines a slot 144 for slidably receiving second member 134 of mount assembly 130. Slot 144 includes a closed distal end 144b and an open proximal end 144a. Slot 144 may be configured for secure engagement to second member 134 of mount assembly 130. Slot 144 may include a coating or be configured with ridges, grooves, or knurls corresponding to those found on first and/or second members 132, 134 that more securely retains guide member 140 to mount assembly 130. Base 142 and slot 144 are configured such that when guide member 140 is secured to mount assembly 130 distal end 142b of guide member 140 is aligned with radial expandable fingers 124 formed in distal end 120b.

Base 142 of guide member 140 further defines a number of openings extending therethrough. Proximal end 142a of guide member 140 defines three openings “A”, “B”, “C”. Distal end 210b of guide member 140 defines a single opening “D”. Openings “A”, “B”, “C” extend into and through base 142 and converge as single opening “D” in distal end 142b of guide member 140. Openings “A”, “B”, “C” are configured to enable a surgeon to secure a screw and/or use a drill or other instrument at three different angles relative to metal plate 20. Center opening “B” is configured perpendicular to metal plate 20, while each of openings “A”, “C” is configured at opposing angles relative to metal plate 20. Openings “A”, “C” allow guide member 140 to be used to install bone screws in a toe-nailed manner as described above. Openings “A”, “C” may be symmetrically formed relative to opening “B”. Opening “A”, “C” may also be configured at any angle relative to a releasably engaged metal plate 20. Because guide member 140 is removable from drill guide 100, guide member 140 may be replaced if it becomes damaged or to better suit the needs of the procedure being performed.

With reference to FIGS. 4A-6B, drill guide 100 releasably connects to metal plate 20 through actuation of handle assembly 110. When handle assembly 110 is in a first or open position, an opening in metal plate 20 receives fingers 124. Articulation of movable handle 116 about handle pin 115 towards fixed handle 114 from a first or open position (FIG. 5A) to a second or closed position (FIG. 6A) causes actuation shaft 126 to advance distally through extension member 120. As described above, engagement of distal end 126b of actuation shaft 126 cause the expansion of radial expandable fingers 124 (FIGS. 4B and 6B).

With continued reference to FIGS. 5A and 6A, in the first or open position (FIG. 5A), movable handle 116 is at maximum separation from fixed handle 114. This open position also corresponds to an unlocked and retracted state of actuation shaft 126. In the first position proximal end 126a of actuation shaft 126 extends beyond latch 119 slidably attached to housing 112 of handle assembly 110. While handle assembly 110 is in the first position latch 119 is also in a first or down position. In this position, proximal end 126a inhibits latch 119 from being slid into a locked position.

When a surgeon squeezes movable handle 116 toward fixed handle 114, distal end 116b of movable handle 116 advances actuation shaft 126 through extension member 120. As leaf springs 118 bias movable handle 116 and fixed handle 114 towards an open position, a surgeon must continue to squeeze movable handle 116 and fixed handle 114 towards each other to maintain the advanced position for shaft 126. To facilitate the use of drill guide 100, however, latch 119 may be used for releasably locking shaft 126 in a nearly fully advanced position. This obviates the need for a surgeon to continue to squeeze handles 114, 116 after advancement of actuation shaft 126 has occurred. Instead, the surgeon's thumb may move latch 119 into abutment with proximal end 126a. Latch 119 remains in place due to the backward pressure applied by proximal end 126a against it. To release handle assembly 110 movable handle 116 is retracted sufficiently to remove the backward pressure applied by proximal end 126a, thus, allowing latch 119 to be returned to a first or down position.

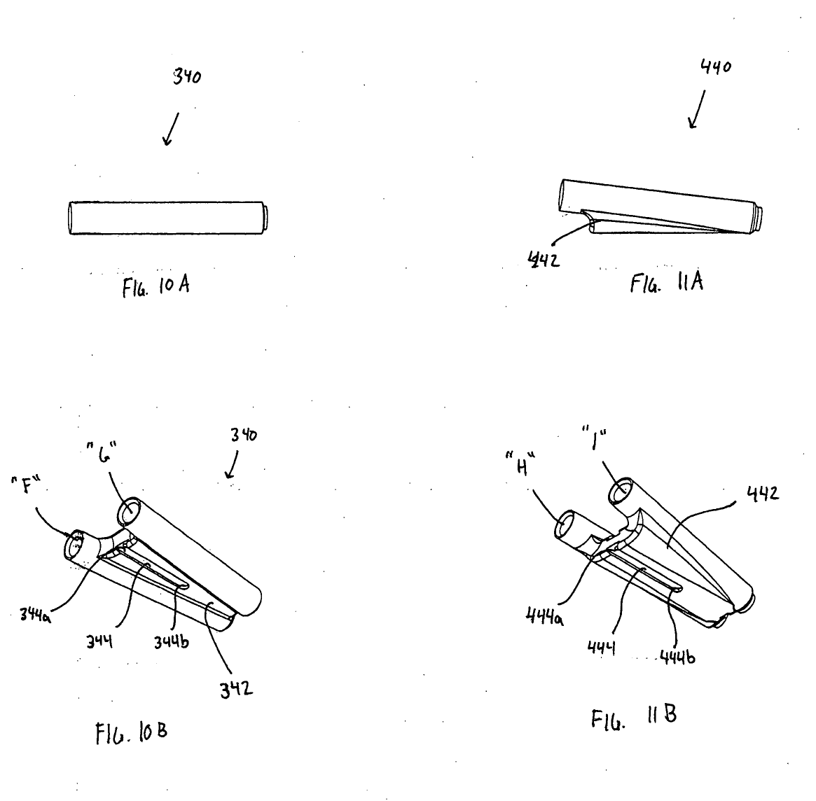

Referring now to FIGS. 9A-11B, shown are alternate embodiments of guide member 140. Guide member 240, 340, 440 are substantially similar to guide member 140. Guide members 240, 340, 440 each include a base 242, 342, 442 defining a slot 244, 344, 444, respectively. Slots 244, 344, 444 each have a proximal end 244a, 344a, 444a, and a distal end 244b, 344b, 444b, respectively. Guide member 240 includes only a single opening “E” extending therethrough, while guide members 340, 440 each include two openings “F”, “G”, “H”, “I”, respectively. Guide members 340, 440 are substantially similar except that slot 344 of guide member 340 is positioned within the same plane as that defined by openings “F”, “G”, while slot 444 of guide member 440 extends in a plane parallel to that formed by openings “H”,

In an alternate embodiment, guide members 140, 240, 340, 440 may include any number of openings sized to receive a drill, bone screw or other instrument. Guide members 140, 240, 340, 440 may include adjustable openings for selectively guiding the drill, bone screw or other instrument.

While there has been described and illustrated specific embodiments of the drill guide apparatus, it will be apparent to those skilled in the art that variations and modifications are possible without deviating from the broad spirit and principle of the present disclosure. The disclosure, therefore shall not be limited to the specific embodiments discussed herein.

What is claimed is:

1. A drill guide apparatus comprising:
a handle assembly;
an extension member rotatably mounted to the handle assembly, the extension member having a distal end configured for releasably engaging an opening in a metal plate;
a mounting assembly affixed to the extension member; and
a guide member releasably attached to the mounting assembly.

2. The apparatus of claim 1, further including an actuation shaft operably disposed within the extension member.

3. The apparatus of claim 2, wherein the distal end of the extension member includes fingers for engaging the metal plate.

4. The apparatus of claim 3, wherein the radially expandable fingers include a lip for engaging the metal plate.

5. The apparatus of claim 3, wherein advancement of the actuation shaft in a distal direction causes radial expansion of the fingers.

6. The apparatus of claim 3, wherein retraction of the actuation shaft causes radial contraction of the fingers.

7. The apparatus of claim 1, wherein the guide member includes one or more openings sized for receiving a drill, bone screw or other instrument.

8. The apparatus of claim 1, wherein the guide member further includes a slot for releasably engaging the mounting assembly.

9. A drill guide apparatus comprising:
a handle assembly including an actuation mechanism, the actuation mechanism configured for distally advancing an actuation shaft; and
an extension member rotatably mounted to the handle assembly configured for slidably receiving the actuation shaft, the extension member including a distal end configured for releasable attachment to a metal plate.

10. The apparatus of claim 9, further including a mounting member affixed to the extension member configured for receiving a guide member.

11. A drill guide apparatus comprising:
a handle assembly;
an extension member extending from the handle assembly, the extension member configured for releasably engaging a metal plate;
a mounting assembly affixed to the extension member; and
a guide member releasably attached to the mounting assembly.

12. The drill guide apparatus of claim 11, wherein the extension member is rotatable attached to the handle assembly.

13. A guide member for guiding a drill, bone screw or other instrument, the member comprising:
a base including one or more openings sized to receive a drill, bone screw or other instrument, the base further including a slot configured for attachment with a drill guide apparatus.

14. The guide member of claim 13, wherein the slot includes an opening proximal end and a closed distal end.

15. The guide member of claim 14, wherein the slot is further configured to be slidingly received by a drill guide apparatus.
